(19) United States
(12) Patent Application Publication
(10) Pub. No.: US 2024/0399052 A1
(43) Pub. Date: Dec. 5, 2024

(54) INFUSION PUMP WITH CAM SHAFT ENCODER

(71) Applicant: B. Braun Medical Inc., Bethlehem, PA (US)

(72) Inventor: Hans Schmolk, Perkasie, PA (US)

(73) Assignee: B. Braun Medical Inc., Bethlehem, PA (US)

(21) Appl. No.: 18/205,195

(22) Filed: Jun. 2, 2023

(51) Int. Cl. A61M 5/142

(52) U.S. Cl. CPC A61M 5/14228; A61M 5/1424; A61M 39/28; A61M 2205/12

(57) ABSTRACT

An ambulatory infusion peristaltic pump has a cam shaft controlled by an encoder magnet assembly including a diametrically polarized magnet and an injection molded plastic housing. The encoder magnet assembly is attached to the cam shaft by a pair of cantilever snaps that are a part of the housing and are keyed to the cams by an alignment pin such that the angular position of the magnet's poles corresponds to the orientation of the cams. The magnetic orientation is detected by an encoder printed circuit board assembly (PCBA) positioned within the ambulatory infusion peristaltic pump and communicates readings to a pump microcontroller that determines the speed of the drive shaft and the angular position of the magnet (and, in turn, the position of each cam) from the PCBA readings and controls the pump based on the determined speed and position data in accordance with pump parameter settings.

### FIELD OF THE INVENTION

The present disclosure is related to infusion pumps and, more particularly, to infusion pumps having a cam shaft that is used to raise and lower fingers of a peristaltic pump that pushes fluid through a tube and an encoder positioned on an end of the cam shaft to accurately determine the position and speed of the cam shaft to precisely deliver fluids through the tube to the patient.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use. These ambulatory pumps are also used in acute clinical settings to physically differentiate routes of therapy (e.g., intravascular, neural/neuraxial, enteral, etc.) so as to reduce possibility of wrong route medication errors.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids is desirable to optimize treatment of a patient as there are many fluids where small variations can be critical.

Infusion pumps have been described that use cam shafts to perform a pump cycle. For example, U.S. Pat. No. 5,131,816 describes a cartridge fed infusion pump containing a plurality of linear peristaltic pumps where each pump is powered by a direct current motor having a cam shaft that rotates to perform a pump cycle. A position encoder mounted on the shaft is used to determine when the cam shaft has reached the stop position in the pump cycle. The pump may have a plurality of fingers or cams that are repeatedly lowered and raised to provide the desired pumping action. Each pump rotates the cam shaft to selectively push against three cam followers or “fingers” including an output valve, a pump plunger, and an input valve. The cam shaft is provided with a timing disk or position encoder. The timing disk is solid except for a sector which is removed. The timing disk can be read by an optical sensor circuit to count the rotations, thereby controlling the rate and location of the cam shaft.

Similarly, in U.S. Pat. No. 6,064,97, a plurality of rotary cams engage a plurality of pumping members, and rotation of the cam shaft causes the pumping members to sequentially act against the tubing to provide a peristaltic pumping action. The movements of a linear peristaltic pump are controlled by an encoder wheel or a volumetric flow equalizing drive control wheel in which the rate of pumping is increased by the shortened time duration for one complete revolution. A timing disk has a plurality of openings around the disk with predetermined spacing therebetween that corresponds to a desired amount of device rotation. A light sensor is operatively positioned adjacent to the encoder wheel for detecting the spaced openings and connected to a switch circuit to provide the electrical signal to turn the motor off each time a next one of the openings is detected by the sensor, thereby allowing the device to traverse the desired amount of device rotation during each regular interval that the motor is turned on.

Also, in U.S. Pat. No. 6,164,921, in addition to an optical sensor, a motor speed control unit includes a shutter or encoder wheel that is attached to a cam shaft and rotatable thereby. The optical sensor and encoder wheel collectively define an optical encoder. The encoder wheel includes four (4) encoder arms extending radially therefrom in equidistantly spaced intervals of approximately 90 degrees. The encoder wheel is oriented relative to the optical sensor such that the encoder arms will intermittently interrupt a beam of light during the rotation of the encoder wheel by the cam shaft. The number and size of the encoder arms is selected such that interruptions in the beam of light caused thereby correspond to pump cycles of the pump, with the optical sensor being operable to determine the beginning and end of each pump cycle and increase the power to the motor and hence the rotational speed of the cam between pump cycles.

Such devices require optical sensors and are relatively complicated and unreliable for use in infusion pumps. Techniques for operating cam shafts in a reliable manner with simplified circuitry remain desired for the precise delivery of fluids to a patient using a peristaltic infusion pump.

### SUMMARY

In sample configurations, an infusion pump is provided that pumps fluid through a tube from a fluid container to a patient using a peristaltic pump having respective pump sliders that are raised and lowered to engage the tube to force fluid through the tube and a cam shaft supporting respective cams adapted to engage with the respective pump sliders to raise and lower the respective pump sliders as the cam shaft rotates. A pump motor turns the cam shaft under control of a controller in accordance with a pump parameter setting. A cam shaft encoder magnet assembly attached to the cam shaft includes a diametrically polarized magnet, and the cam shaft encoder magnet assembly is configured to sense a magnetic orientation of the diametrically polarized magnet as the cam shaft rotates. The controller receives magnetic orientation data from the cam shaft encoder magnet assembly, processes the received magnetic orientation data to calculate a speed and angular position of the cam shaft, and controls the pump motor driving the camshaft in response to the calculated speed and angular position of the cam shaft to raise and lower the respective pump sliders in accordance with a pump parameter setting.

In the sample configurations, the cam shaft encoder magnetic assembly is housed in an injection molded plastic housing. The injection molded plastic housing includes a pair of cantilever snaps that connect the injection molded plastic housing to the cam shaft. An alignment pin keys the pair of cantilever snaps to the respective cams such that an angular position of poles of the diametrically polarized magnet correspond to orientation of the respective cams. The cam shaft encoder magnet assembly may further include an encoder printed circuit board that detects the magnetic orientation of the diametrically polarized magnet and provides the magnetic orientation data to the controller.

The following description also describes a method of operating an infusion pump to pump a fluid through a tube to a patient. In sample configurations, the method includes rotating a cam shaft in accordance with a pump parameter setting, the cam shaft supporting respective cams adapted to engage with the respective pump sliders to raise and lower the respective pump sliders as the cam shaft rotates, wherein the pump sliders engage with the tube to provide fluid from a fluid container to a patient as the respective pump sliders are raised and lowered to force fluid through the tube. The method further includes sensing, using a cam shaft encoder magnet assembly attached to the cam shaft, a magnetic orientation of the cam shaft as the cam shaft rotates, processing sensed magnetic orientation data to calculate a speed and angular position of the cam shaft, and adjusting rotation of the cam shaft to adjust the speed and angular position of the cam shaft to raise and lower the respective pump sliders in accordance with a pump parameter setting.

In the sample configurations, the method includes sensing the magnetic orientation of the cam shaft as the cam shaft rotates by sensing the magnetic orientation of a diametrically polarized magnet attached to the cam shaft. Sensing the magnetic orientation of the cam shaft may be performed by an encoder printed circuit board that further provides the magnetic orientation data to the controller to control rotation of the cam shaft in accordance with the pump parameter setting.

The method may further include housing the cam shaft encoder magnet assembly in an injection molded plastic housing connecting the injection molded plastic housing to the cam shaft using a pair of cantilever snaps and keying the pair of cantilever snaps to the cams with an alignment pin such that an angular position of poles of the diametrically polarized magnet correspond to orientation of the respective cams.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a perspective view of an example ambulatory peristaltic infusion pump.

FIG. 2 is a perspective view of an example infusion cassette with a free flow prevention clamp for use with the ambulatory peristaltic infusion pump of FIG. 1.

FIG. 3 is a partial perspective view of the ambulatory peristaltic infusion pump of FIG. 1.

FIGS. 4 and 5 are cutaway views of the ambulatory peristaltic infusion pump of FIG. 1 illustrating pump sliders and cam rods for moving the pump sliders.

FIG. 6 is a diagram illustrating the pump microcontroller that operates the ambulatory peristaltic infusion pump in a sample configuration.

FIG. 7 is a perspective view of the cam shaft encoder of the ambulatory peristaltic infusion pump shown in FIG. 5.

FIG. 8 is a flow chart illustrating the operation of the cam shaft encoder of FIG. 7 in a sample configuration.

FIG. 9 is a functional block diagram illustrating a general-purpose computer hardware platform configured to implement the functional examples described with respect to FIGS. 1-8.

FIG. 10 is another functional block diagram illustrating a general-purpose computer hardware platform configured to implement the functional examples described with respect to FIGS. 1-8.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the cam shaft encoder described herein may be used with a variety of other pump types.

An infusion pump having a cam shaft controlled by an encoder magnet assembly is described. The encoder magnet assembly includes a diametrically polarized magnet and an injection molded plastic housing where the encoder magnet assembly is attached to the cam shaft by a pair of cantilever snaps that are a part of the housing and are keyed to the cams by an alignment pin such that the angular position of the magnet's poles corresponds to the orientation of the cams. The magnetic orientation is detected by an encoder printed circuit board assembly (PCBA) positioned within the pump adjacent the peristaltic pump where readings from the PCBA are communicated to a pump microcontroller. The pump microcontroller determines the speed of the drive shaft and the angular position of the magnet (and, in turn, the position of each cam) from the PCBA readings and controls the pump based on the determined speed and position data in accordance with pump parameter settings.

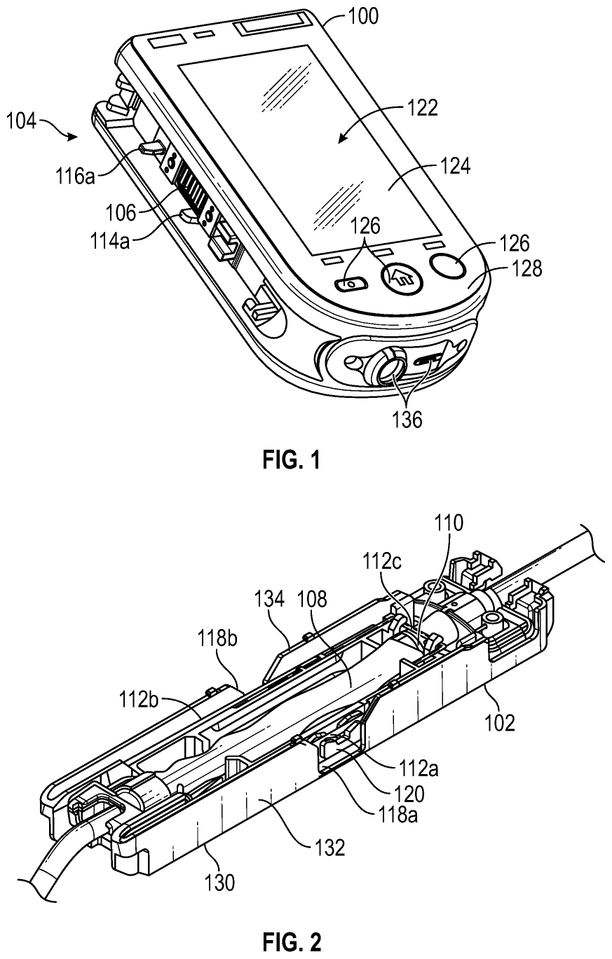

FIG. 1 depicts an example ambulatory peristaltic infusion pump 100, while FIG. 2 depicts an example infusion cassette 102 for use with the ambulatory peristaltic infusion pump 100. As illustrated in FIG. 1, the ambulatory peristaltic infusion pump 100 includes a receptacle 104 configured to receive the infusion cassette 102. During operation, the user inserts the infusion cassette 102 part way into the receptacle 104 of the ambulatory peristaltic infusion pump 100. The user closes a latch of the ambulatory peristaltic infusion pump 100, thus pulling the infusion cassette 102 fully flush into the receptacle 104. When software of the infusion pump 100 detects that the latch is closed (e.g., via a Hall effect sensor), the software checks the pressure of tubing of the installed infusion cassette 102 to ensure it is above a predetermined threshold. If the pressure sensors sense a sufficiently high pressure, then it is determined that an infusion cassette 102 is physically present. At this point, a cassette ID algorithm is invoked that uses the cassette ID sensors to read all the markings on the side of the cassette 102 to then determine the cassette ID value. The cassette ID value is then passed along to user interface software of the pump 100 to determine how to use the cassette ID for controlling pump parameters.

A peristaltic pump mechanism 106 within the receptacle 104 acts upon a tube 108 extending through a channel within the infusion cassette 102 to pump fluid from a fluid container (e.g., a bag or a bottle; not shown) into a patient. An example free flow prevention clamp 110 is positioned within the infusion cassette 102 to allow fluid flow through the tube 108 when the infusion cassette 102 is coupled to the ambulatory peristaltic infusion pump 100 within the receptacle 104 (during which time the peristaltic pump mechanism 106 controls fluid flow through the tube 108) and to selectively cut off fluid flow through the tube 108 when the infusion cassette 102 is not coupled to the ambulatory peristaltic infusion pump 100 in order to prevent unintentional fluid flow through the tube 108 (e.g., free flow).

The ambulatory peristaltic infusion pump 100 includes a user interface 122 for interacting with the ambulatory peristaltic infusion pump 100. The illustrated user interface 122 includes a display 124 (which may be a touchscreen) and buttons 126. A user controls the operation of the ambulatory peristaltic infusion pump 100 via the user interface 122. The ambulatory peristaltic infusion pump 100 additionally includes a housing 128 for containing and supporting the components of the ambulatory peristaltic infusion pump 100 such as the peristaltic pump mechanism 106, electronics, power supplies, and the like.

The free flow prevention clamp 110 includes a first elongate section 112a, a second elongate section 112b, and a clamping section 112c. The housing 130 of the infusion cassette 102 supports the free flow prevention clamp 110. The clamping section 112c is positioned within the cassette geometry such that, when the infusion cassette 102 is received within the receptacle 104 of the ambulatory peristaltic infusion pump 100, the clamping section 112c extends across the channel receiving the tube 108. The housing 130 of the infusion cassette 102 may be rigid plastic or other material capable of supporting the tube 108 and free flow prevention clamp 110.

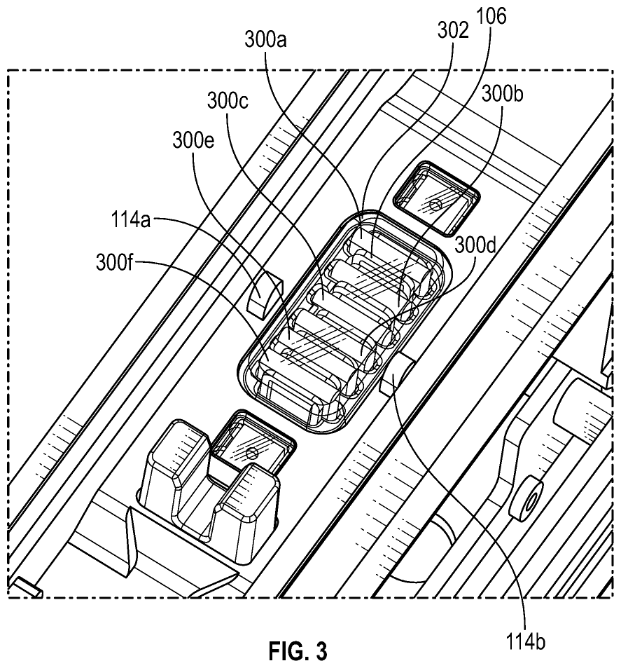

The ambulatory peristaltic infusion pump 100 also includes a pair of arc cams 114a and 114b (FIG. 3). The first arc cam 114a is shown on one side of the receptacle illustrated in FIG. 1, but the second arc cam 114b is hidden from view. The pair of arc cams 114a and 114b engage the elongate sections 112a, 112b of the free flow prevention clamp 110 in order to lift the clamping section 112c. Additionally, the ambulatory peristaltic infusion pump 100 includes a pair of wedge cams 116a and 116b. A first wedge cam 116a is shown on one side of the receptacle 104 illustrated in FIG. 1, but the second wedge cam 116b is hidden from view. The pair of wedge cams 116a and 116b transition the free flow prevention clamp 110 from an open, manufactured/shipped state to an operational state, which is described in further detail below.

As shown in FIG. 2, the infusion cassette 102 also includes a first cutout 118a in a sidewall 132 of the infusion cassette 102 and a second cutout 118b in an opposite sidewall 134 of the infusion cassette 102. Additionally, the infusion cassette 102 includes a bypass button/pad 120 positioned on the first elongate section 112a adjacent a mid-point of the first elongate section 112a and the first cutout 118a. The bypass button/pad 120 and cutout 118a together facilitate engagement of the first elongate section 112a by a finger of an operator in order to manually lift the clamping section 112c to allow fluid flow through the tube 108 (e.g., for priming the infusion cassette 102) when the infusion cassette 102 is not received within the receptacle 104 of the ambulatory peristaltic infusion pump 100. The bypass button/pad 120 may be a press fit piece of rigid plastic. Although the bypass button/pad 120 is illustrated as only on the first elongate section 112a, the bypass button/pad 120 also may be provided on the second elongate section 112b.

The ambulatory infusion pump 100 further includes connector ports 136 that provide electronic access for control and for powering the ambulatory peristaltic infusion pump 100 when used in the configurations described below.

FIG. 3 depicts the peristaltic pump mechanism 106 of the ambulatory peristaltic infusion pump 100. The peristaltic pump mechanism 106 includes multiple pump sliders 300 (six pump sliders 300a-f illustrated in FIG. 3). A flexible barrier (seal) 302 separates the pump sliders 300 (and other pump components of a pumping mechanism) from the receptacle 104 receiving the infusion cassette 102 with the tube 108. The flexible barrier 302 provides a barrier between the fluid delivery apparatus/cassette and the pumping mechanism to prevent errant/leaked fluid from damaging internal components of the pumping mechanism.

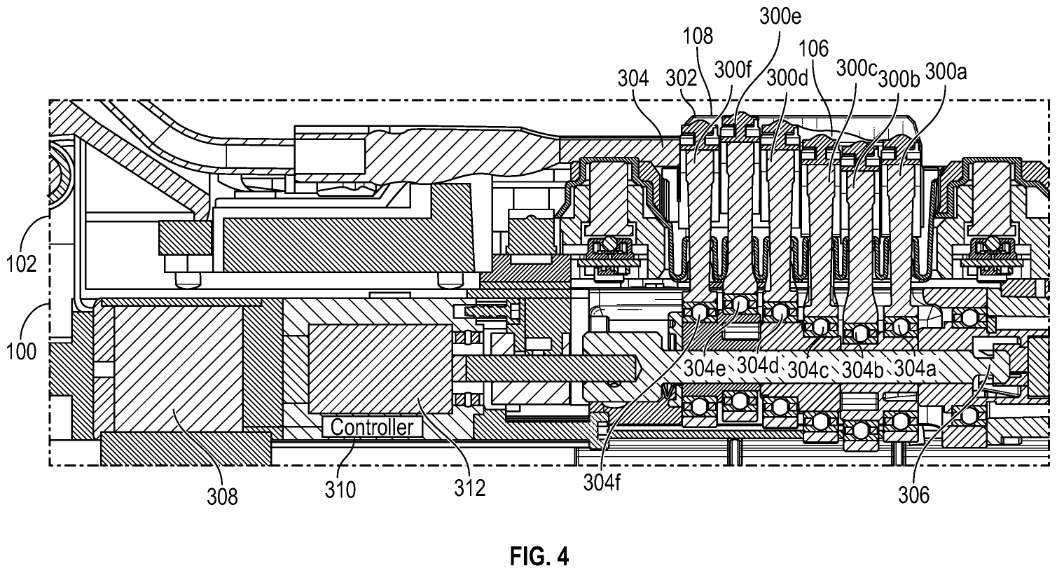

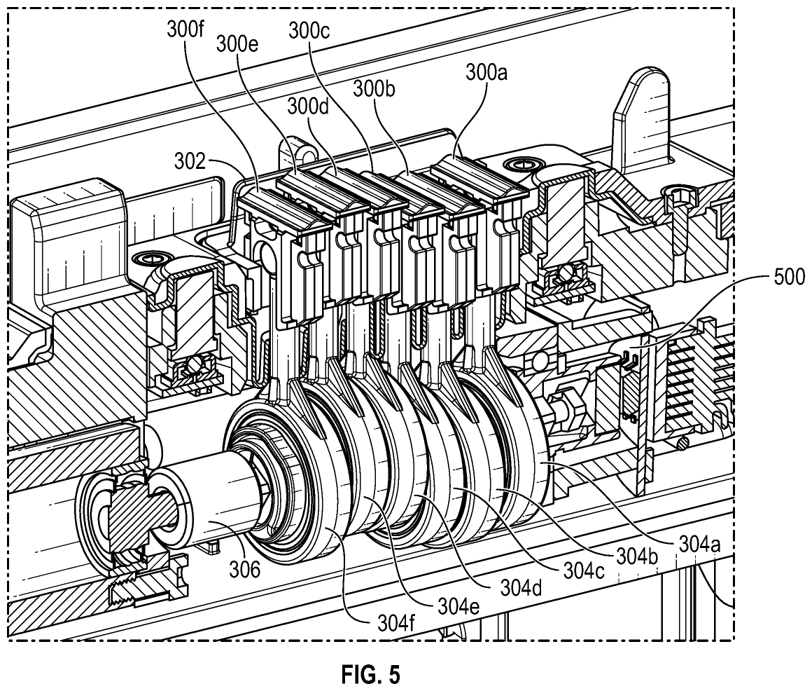

FIGS. 4 and 5 are cutaway views of the ambulatory peristaltic infusion pump 100 with the infusion cassette 102 inserted into the receptacle 104 of the ambulatory peristaltic infusion pump 100. Multiple cams 304 (six cams 304a-f) supported by a cam shaft 306 of the ambulatory peristaltic infusion pump 100 act on respective pump sliders 300a-300f The cams 304a-304f respectively raise and lower the pump sliders 300a-300f, which engage the tube 108 of the infusion cassette 102 in order to force fluid though the tube 108. A pump motor 308 under control of a controller 310 turns the cam shaft 306 by way of a gearbox 312. As the cam shaft 306 turns, the cams 304a-300f, which are offset from each other in an axial direction, raise and lower respective pump sliders 300a-300f For example, cam 304a raises and lowers pump slider 300a; cam 304b raises and lowers pump slider 300b, and the like. The controller 310 may be a standalone or embedded processor configured to carry out instructions in order to control operations of the ambulatory peristaltic infusion pump 100.

The controller 310 may include a main controller such as a dual core 32 bit processor from NXP of Eindhoven, Netherlands (e.g., model #MCIMX7S5EVM08SC), a microcontroller from NXP (e.g., model #MKV31F512VLH12), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump cam shaft revolutions per minute (RPM) corresponding to the infusion flow rate from a system control core of the main processor. The microcontroller develops a pulse width modulation (PWM) motor drive parameter relating to the desired cam shaft RPM. The PWM output of the microcontroller becomes the motor drive input to the pump motor driver, which contains motor drive transistors and protection circuitry. The rotation of the cam shaft 306 of the pumping mechanism is measured by the magnetic encoder. At specified time intervals, the output of the magnetic encoder is read by the microcontroller, which uses the encoder value to compute the speed of the cam shaft 306 and the position of the pump rotation. These values are then used to modify the PWM output to maintain the correct cam shaft RPM.

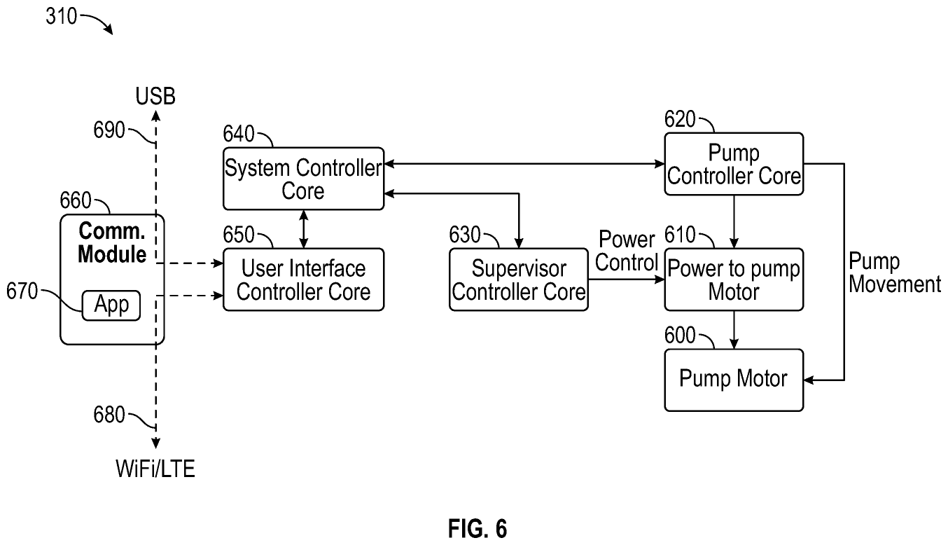

In sample configurations, the pump controller 310 includes multiple cores designed to ensure safe operation of the ambulatory peristaltic infusion pump 100. As described above with respect to FIGS. 1-5, the ambulatory peristaltic infusion pump 100 includes a peristaltic pump 106 configured to deliver fluid (e.g., pain medication) to a patient in response to pump controller 310. FIG. 6 is a diagram illustrating the entire microcontroller network within the peristaltic pump 106. As shown, the microcontroller network includes communication module 660 and multiple controller cores 620-650 used as pump controller 310 that together ensure safe operation of the peristaltic pump 106 in sample configurations. The peristaltic pump 106 includes a pump motor 600 that is powered by a power source 610 and controlled by a collection of interconnected controller cores 620-650 that monitor operation of each other and process communications within the peristaltic pump 106. For example, the system controller core 640 communicates to the pump controller core 620 the parameters for pumping and the pump controller core 620 controls the power source 610 to -selectively power the pump motor 600.

In sample configurations, all communications with the ambulatory peristaltic infusion pump 100 pass through a communications module (“comm module”) 660 that processes communications within the ambulatory peristaltic infusion pump 100. The comm module 660 is capable of wireless communication and wired communication. Software safeguards are implemented by a communications application (“comm app”) 670 as well as pump side applications that validate the USB (Universal Serial Bus) communication path to prevent unauthorized access to the ambulatory peristaltic infusion pump 100 via the comm module 660 that could interfere with proper operation.

In the configuration illustrated in FIG. 6, the pump controller core 620 is dedicated to control movement of the pump motor 600. The pump controller core 620 may be located on a circuit board to which the pump motor 600 is electrically connected. The pump core controller 620 is responsible for controlling movement of the pump motor 600 (e.g., start/stop/rotation rate/etc.). No other controller core has direct access to the pump motor 600 (with the limited exception of the supervisor controller core 630). The power to pump motor 600 is provided by a pump motor driver (not shown). The supervisor controller core 630 controls overall power to the pump motor driver, but the pump controller core 620 also has the ability to enable/disable the pump motor driver, which in turn provides the power to the pump motor 600.

The supervisor controller core 630 is dedicated to supervisory operations for the pump motor 600. The supervisor controller core 630 can stop the pump motor 600 by, for example, controlling the power to the pump motor at power source 610 in the event any one of the other controller cores 620, 640, or 650 is determined to be behaving improperly. The supervisor controller core 630 monitors the health of all other controller cores in the ambulatory peristaltic infusion pump 100. In the event that a controller core is behaving improperly, the supervisor controller core 630 is capable of stopping power flow to the pump motor 600 by means of switching off the power source 610 to the pump motor 600. It will be appreciated that the pump motor 600 may be stopped without cutting all power to the pump motor 600.

The system controller core 640 and user interface controller core 650 control other operations of the ambulatory peristaltic infusion pump 100 (e.g., graphical user input/output, infusion state logic, alarms, configurations, etc.). Controller cores 640 and 650 are interconnected with each other. The system controller core 640 is interconnected with the pump controller core 620 and the supervisor controller core 630. As noted above, if not explicitly notified of a problem, the supervisor controller core 630 monitors the other controller cores for improper behavior and acts as appropriate.

The comm module 660 includes the comm app 670 that implements a wireless interface 680 and wired interface 690 through which wireless and wired communications must pass. The comm app 670 checks certificates from external wireless signal sources that use the wireless communications interface 680 to ensure the external signal source is authorized to communicate with the ambulatory peristaltic infusion pump 100 and that the certificates are valid (e.g., current and have not been revoked). Pump applications check the certificates on the wired interface path. Communications with the ambulatory peristaltic infusion pump 100 are only possible with a valid certificate. Messages from the wired interface 690 also may be checked for valid certificates, but internal messages from known sources within the ambulatory peristaltic infusion pump 100 need not require such certificates provided physical access to the controller hardware is prevented.

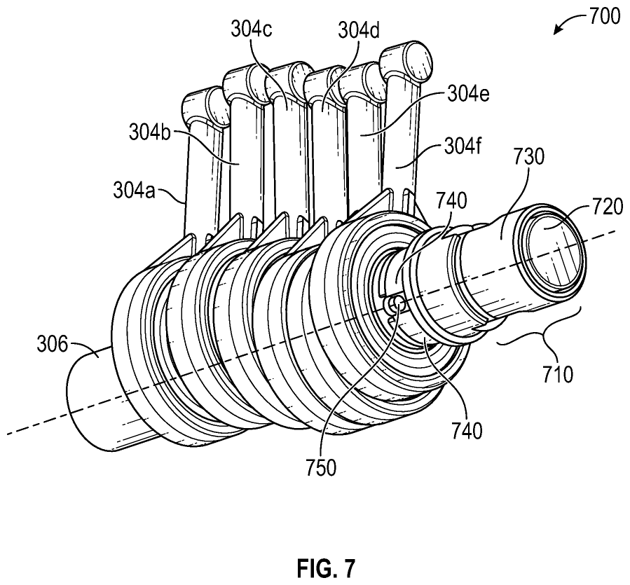

FIG. 7 is a perspective view of the cam shaft encoder 700 of the ambulatory peristaltic infusion pump 100 shown in FIG. 5. As shown in FIG. 5, the cam shaft 306 within the ambulatory peristaltic infusion pump 100 supports cams 304a-304f that raise and lower corresponding pump sliders 300a-300f as the cam shaft 306 turns. An encoder printed circuit board assembly (PCBA) 500 also may be positioned adjacent to the peristaltic pump mechanism to determine the speed of the cam shaft 306 and the angular position of each cam 304a-304f. As shown in FIG. 7, the cam shaft 306 includes an encoder magnet assembly 710 including a diametrically polarized magnet 720 and an injection molded plastic housing 730. Diametrically polarized magnets 720 have poles on opposite sides of the magnet diameter face and are conventionally used for magnetic angle and rotation sensing. Such a magnetization direction differs from conventional axially-magnetized disk magnets that have the north pole on one face and the south pole on the other. The encoder magnet assembly 710 is attached to the cam shaft 306 by a pair of cantilever snaps 740 that are a part of the housing 730 and are keyed to the cams 304a-304f by an alignment pin 750 such that the angular position of the poles of the diametrically polarized magnet 720 corresponds to the orientation of the cams 304a-304f.

The magnetic orientation of the diametrically polarized magnet 720 is detected by the PCBA 500 positioned within the ambulatory peristaltic infusion pump 100 adjacent to the peristaltic pump mechanism 106. Readings from the PCBA are communicated to the pump controller core 620 (FIG. 6), which determines the speed of the cam shaft 306 and the angular position of the diametrically polarized magnet 720 (and, in turn, the position of each cam 304a-304f) with a high degree of accuracy. The speed of the cam shaft 306 and the angular position of the diametrically polarized magnet 720 are used by the pump controller core 620 to regulate the operation of the infusion pump 100 in accordance with a pump parameter setting.

It will be appreciated that the use of the cam shaft encoder 700 eliminates the need for an optical encoder and the associated optics and optical sensors. Magnetic encoders are used in sample embodiments as magnetic encoders are cost effective solutions that may take the place of optical encoders and the associated optics and optical sensors. Magnetic encoders also are ideal for space constrained designs and low power battery-operated applications. The motor assembly consists of a motor and gearbox which reduces the motor output 35:1 (35 motor revolutions to 1 camshaft revolution). The magnetic encoder on the camshaft 700 in this application accurately measures the speed of the pumping mechanism to provide a 1 to 1 correlation of pumping speed to the microcontroller 310.

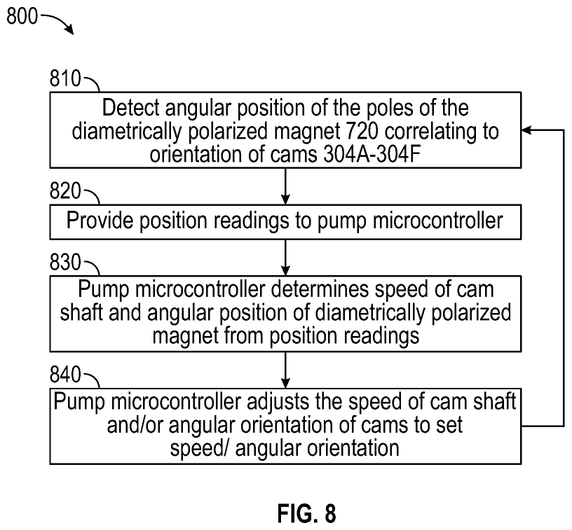

FIG. 8 is a flow chart 800 illustrating the operation of the cam shaft encoder of FIG. 7 in a sample configuration. As noted above, during operation of the peristaltic pump mechanism 106, the cam shaft 306 is rotated to cause the cams 304a-304f to raise and lower the pump sliders 300a-300f, which engage the tube 108 of the infusion cassette 102 in order to force fluid though the tube 108. The pump motor 308 under control of the controller 310 turns the cam shaft 306 by way of a gearbox 312. As the cam shaft 306 turns, the cams 304a-300f, which are offset from each other in an axial direction, raise and lower the respective pump sliders 300a-300f The angular position of the poles of the diametrically polarized magnet 720 corresponds to the orientation of the respective cams 304a-304f, whereby a determination of the magnetic orientation of the poles of the diametrically polarized magnet 720 is representative of the angular position of the cam shaft 306 and hence of the cams 304a-304f. At 810, the PCBA 500 provides readings of the angular position of the poles of the diametrically polarized magnet 720, in the form of incremental quadrature logic signals to the pump controller core 620. At 820, the pump controller core 620 calculates the readings from the PCBA 500. At 830, the readings from the PCBA 500 are used by the pump controller core 620 to determine the speed of the cam shaft 306 and the angular position of the diametrically polarized magnet 720 (and, in turn, the position of each cam 304a-304f) relative to position. The speed of the cam shaft 306 and the angular position of the diametrically polarized magnet 720 may be fed back to the pump controller core 620 at 840 to adjust (i.e., regulate) the operation of the infusion pump 100 in accordance with one or more pump parameter settings.

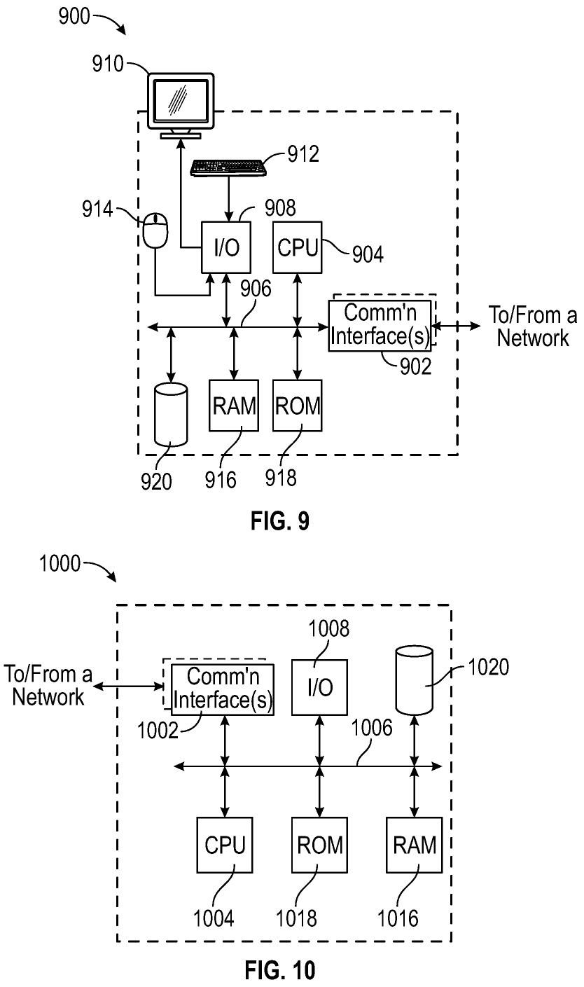

FIGS. 9 and 10 are functional block diagrams illustrating general-purpose computer hardware platforms configured to implement the functional examples described above with respect to FIGS. 1-8.

Specifically, FIG. 9 illustrates an example computer platform 900 and FIG. 10 depicts an example computer 1000 with user interface elements, as may be used to implement in a personal computer, ambulatory peristaltic infusion pump 100, or other type of workstation or terminal device. It is believed that those skilled in the art are familiar with the structure, programming and general operation of such computer equipment and as a result the drawings should be self-explanatory.

The hardware of an example processing device (e.g., computer) 900 (FIG. 9) includes a data communication interface 902 for packet data communication. The computer 900 also includes a central processing unit (CPU) 904, in the form of circuitry forming one or more processors, for example, the controller 310 for executing program instructions. The computer platform hardware typically includes an internal communication bus 906, program and/or data storage 916, 918, and 920 for various programs and data files to be processed and/or communicated by the computer 900, although the computer 900 often receives programming and data via network communications. In one example, as shown in FIG. 9, the computer 900 may include a video display unit 910, (e.g., a liquid crystal display (LCD) or other display 124 of the ambulatory peristaltic pump device 100), an input device 912 (e.g. a keyboard or user interface 122 and/or buttons 126), and an optional cursor control device 914 (e.g. a mouse), each of which communicate via an input/output device (I/O) 908. The hardware elements, operating systems, and programming languages of such computers 900 are conventional in nature, and it is presumed that those skilled in the art are adequately familiar therewith. Of course, the computer functions may be implemented in a distributed fashion on a number of similar hardware platforms, to distribute the processing load.

Hardware of a user terminal device 1000, such as a PC (Personal Computer) or tablet computer, similarly includes a data communication interface 1002, CPU 1004, main memory 1016 and 1018, one or more mass storage devices 1020 for storing user data and the various executable programs, an internal communication bus 1006, and an input/output device (I/O) 1008 (see FIG. 10).

Aspects of the methods for pump control, as outlined above, may be embodied in programming in general purpose computer hardware platforms (such as described above with respect to FIGS. 9 and 10), e.g., in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another, for example, from a processor 904 of the computer 900 and/or from a controller 310 or 620 of an ambulatory peristaltic infusion pump 100 to a computer or software of another system (not shown). Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables; copper wire and fiber optics, including the wires that include a bus within a computer system. Carrier-wave transmission media can take the form of electric or electromagnetic signals, or acoustic or light waves such as those generated during radio frequency (RF) and light-based data communications. Common forms of computer-readable media therefore include for example: a floppy disk, a flexible disk, hard disk, magnetic tape, any other magnetic medium, a CD-ROM (compact disc read only memory), DVD (Digital Video Disks) or DVD-ROM, any other optical medium, punch cards paper tape, any other physical storage medium with patterns of holes, a RAM (Random Access Memory), a PROM (Programmable Read Only Memory) and EPROM (Electrically Programmable Read Only Memory), a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render computer system or device into a special-purpose machine that is customized to perform the operations specified in the program performed by the controller 310 or 620 of the ambulatory peristaltic infusion pump 100.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump that provides fluid from a fluid container to a patient via a tube, comprising:
a peristaltic pump comprising respective pump sliders that are raised and lowered to engage the tube to force fluid through the tube, a cam shaft supporting respective cams adapted to engage with the respective pump sliders to raise and lower the respective pump sliders as the cam shaft rotates, and a pump motor that turns the cam shaft;
a cam shaft encoder magnet assembly attached to the cam shaft, the cam shaft encoder magnet assembly comprising a diametrically polarized magnet, the cam shaft encoder magnet assembly configured to sense a magnetic orientation of the diametrically polarized magnet as the cam shaft rotates; and
a controller that receives magnetic orientation data from the cam shaft encoder magnet assembly, processes the received magnetic orientation data to calculate a speed and angular position of the cam shaft, and controls the pump motor driving the cam shaft in response to the calculated speed and angular position of the cam shaft to raise and lower the respective pump sliders in accordance with a pump parameter setting.

2. The infusion pump of claim 1, further comprising an injection molded plastic housing that houses the cam shaft encoder magnet assembly.

3. The infusion pump of claim 2, wherein the injection molded plastic housing comprises a pair of cantilever snaps that connect the injection molded plastic housing to the cam shaft.

4. The infusion pump of claim 3, further comprising an alignment pin that keys the pair of cantilever snaps to the respective cams such that an angular position of poles of the diametrically polarized magnet correspond to orientation of the respective cams.

5. The infusion pump of claim 1, wherein the cam shaft encoder magnet assembly comprises an encoder printed circuit board that detects the magnetic orientation of the diametrically polarized magnet.

6. The infusion pump of claim 5, wherein the encoder printed circuit board further provides the magnetic orientation data to the controller.

7. A method of pumping a fluid through an infusion pump, comprising:
rotating a cam shaft in accordance with a pump parameter setting, the cam shaft supporting respective cams adapted to engage with respective pump sliders to raise and lower the respective pump sliders as the cam shaft rotates;
the pump sliders engaging with a tube adapted to provide fluid from a fluid container to a patient as the respective pump sliders are raised and lowered to force fluid through the tube;
sensing, using a cam shaft encoder magnet assembly attached to the cam shaft, a magnetic orientation of the cam shaft as the cam shaft rotates;
processing sensed magnetic orientation data to calculate a speed and angular position of the cam shaft; and
adjusting rotation of the cam shaft to adjust the speed and angular position of the cam shaft to raise and lower the respective pump sliders in accordance with a pump parameter setting.

8. The method of claim 7, wherein sensing the magnetic orientation of the cam shaft as the cam shaft rotates comprises sensing the magnetic orientation of a diametrically polarized magnet attached to the cam shaft.

9. The method of claim 8, further comprising housing the cam shaft encoder magnet assembly in an injection molded plastic housing.

10. The method of claim 9, further comprising connecting the injection molded plastic housing to the cam shaft using a pair of cantilever snaps.

11. The method of claim 10, further comprising keying the pair of cantilever snaps to the cams with an alignment pin such that an angular position of poles of the diametrically polarized magnet correspond to orientation of the respective cams.

12. The method of claim 8, wherein sensing the magnetic orientation of the cam shaft comprises detecting, using an encoder printed circuit board, the magnetic orientation of the diametrically polarized magnet.

13. The method of claim 12, further comprising providing the magnetic orientation data to a controller that controls rotation of the cam shaft in accordance with the pump parameter setting.

14. A non-transitory controller-readable storage medium storing controller-executable instructions that, when executed by a controller of an infusion pump cause the infusion pump to perform operations comprising:
rotating a cam shaft in accordance with a pump parameter setting, the cam shaft supporting respective cams adapted to engage with respective pump sliders to raise and lower the respective pump sliders as the cam shaft rotates, the pump sliders engaging with a tube adapted to provide fluid from a fluid container to a patient as the respective pump sliders are raised and lowered to force fluid through the tube;
receiving, from a cam shaft encoder magnet assembly attached to the cam shaft, a magnetic orientation of the cam shaft as the cam shaft rotates;
processing sensed magnetic orientation data to calculate a speed and angular position of the cam shaft; and
adjusting rotation of the cam shaft to adjust a speed and angular position of the cam shaft to raise and lower the respective pump sliders in accordance with a pump parameter setting.

15. The storage medium of claim 14, further storing computer-executable instructions that, when executed by the controller of the infusion pump cause the infusion pump to sense the magnetic orientation of a diametrically polarized magnet attached to the cam shaft.

16. The storage medium of claim 15, further storing computer-executable instructions that, when executed by the controller of the infusion pump cause the infusion pump to receive, from an encoder printed circuit board, the magnetic orientation of the diametrically polarized magnet and to process the received magnetic orientation data.
